SEQUENCE LISTING (14 sequences)

SEQ ID NO: 1 - DNA, 41 bp, SARS-CoV-2
  source 1..41: mol_type = genomic DNA
tgcggccaatgtttgtaatcagccaaggaaattttggggac

SEQ ID NO: 2 - DNA, 39 bp, SARS-CoV-2
  source 1..39: mol_type = genomic DNA
cgcattggcatggaagtcactttgatggcacctgtgtag

SEQ ID NO: 3 - DNA, 18 bp, SARS-CoV-2
  source 1..18: mol_type = genomic DNA
aacacaagctttcggcag

SEQ ID NO: 4 - DNA, 22 bp, SARS-CoV-2
  source 1..22: mol_type = genomic DNA
gaaatttggatctttgtcatcc

SEQ ID NO: 5 - DNA, 19 bp, SARS-CoV-2
  source 1..19: mol_type = genomic DNA
ttccttgtctgattagttc

SEQ ID NO: 6 - DNA, 18 bp, SARS-CoV-2
  source 1..18: mol_type = genomic DNA
accttcgggaacgtggtt

SEQ ID NO: 7 - DNA, 39 bp, Homo sapiens
  source 1..39: mol_type = genomic DNA
tggaccctgaagactcggttttagccactgactcggatc

SEQ ID NO: 8 - DNA, 45 bp, Homo sapiens
  source 1..45: mol_type = genomic DNA
cctccgtgatatggctcttcgtttttttcttacatggctctggtc

SEQ ID NO: 9 - DNA, 17 bp, Homo sapiens
  source 1..17: mol_type = genomic DNA
ttgatgagctggagcca

SEQ ID NO: 10 - DNA, 18 bp, Homo sapiens
  source 1..18: mol_type = genomic DNA
caccctcaatgcagagtc

SEQ ID NO: 11 - DNA, 20 bp, Homo sapiens
  source 1..20: mol_type = genomic DNA
atgtggatggctgagttgtt

SEQ ID NO: 12 - DNA, 20 bp, Homo sapiens
  source 1..20: mol_type = genomic DNA
catgctgagtactggacctc

SEQ ID NO: 13 - DNA, 10 bp, SARS-CoV-2
  source 1..10: mol_type = genomic DNA
attggtcgca

SEQ ID NO: 14 - DNA, 11 bp, Homo sapiens
  source 1..11: mol_type = genomic DNA
tcagggtcaca

(19) United States
(12) Patent Application Publication
(10) Pub. No.: US 2024/0229167 A9
(43) Pub. Date: Jul. 11, 2024

(54) METHOD FOR RAPIDLY AND ACCURATELY DETECTING SARS-CoV-2 NUCLEIC ACID

(71) Applicant: Colorado Genomics LLC, Fort Collins, CO (US)

(72) Inventors: Tassa K. Saldi, Erie, CO (US); Erika L. Lasda, Westminster, CO (US); Alfonso Garrido-Lecca, Erie, CO (US); Patrick K. Gonzales, Fort Collins, CO (US)

(73) Assignee: Colorado Genomics LLC, Fort Collins, CO (US)

(21) Appl. No.: 18/336,359

(22) Filed: Jun. 16, 2023

Related U.S. Application Data
(63) Continuation of application No. PCT/US2021/063898, filed on Dec. 16, 2021.
(60) Provisional application No. 63/126,539, filed on Dec. 17, 2020.

(51) Int. Cl. C12Q 1/70; C12Q 1/34; C12Q 1/6844

(52) U.S. Cl. CPC C12Q 1/701; C12Q 1/34; C12Q 1/6846; G01N 2333/165

(57) ABSTRACT

Methods and a kit for detection of genetic material from SARS-CoV-2 that combines reverse transcription loop-mediated isothermal amplification (RT-LAMP) technology with specific oligonucleotide primers, fluorophore-labeled oligonucleotides, quencher technology, buffer components, enzymes, and enzyme ratios chosen to minimize false positive and false negative results, are described. The method includes internal positive control targeting sequences, allowing more certain interpretation of the results. The reaction can be performed at a single elevated temperature, can be completed in 1-2 hours, and the results can readily be interpreted by visually observing the fluorescence color of the reaction using ultraviolet light.

### CROSS-REFERENCE TO RELATED APPLICATIONS

The present application claims the benefit of U.S. Provisional Patent Application No. 63/126,539 for “Method To Rapidly And Accurately Detect Pathogen Nucleic Acid” which was filed on 17 Dec. 2020, the entire content of which patent application is hereby specifically incorporated by reference herein for all that it discloses and teaches.

### INCORPORATION BY REFERENCE OF THE SEQUENCE LISTINGS

The accompanying XML Sequence Listing named (<ST26SequenceListing dtdVersion=“V1_3” fileName=“TUMI01USC1.xml” softwareName=“WIPO Sequence” softwareVersion=“2.3.0” productionDate=“2023-12-11”>Size=13.0 KB) submitted on Dec. 16, 2023 in U.S. patent application Ser. No. 18/336,339 is hereby incorporated by reference.

### BACKGROUND

There are a number of solutions for the detection of SARS-CoV-2/COVID-19 and other pathogenic infections caused by viral, bacterial, fungal, or protozoan microorganisms. Some of these solutions attempt to provide highly accurate or highly sensitive results using molecular analysis such as PCR, but these solutions fail to meet the needs of the industry because of their reliance on expensive laboratory equipment, skilled technicians, and complex lengthy protocols, resulting in excessive turnaround time for receiving results. Other solutions attempt to detect SARS-CoV-2 and other pathogenic infections quickly with rapid turnaround time, using antigen testing which can return results in a few minutes, but these solutions are similarly unable to meet the needs of the industry because of the high degree of false negative results produced by antigen testing technology. Still other solutions must use invasive or painful nasal, nasopharyngeal, or throat swabs to obtain a biological sample for detecting SARS-CoV-2 and other pathogenic infections, but these solutions also fail to meet industry needs because of the patient discomfort and non-compliance associated with these swabs, the reliance on skilled medical professionals for proper collection, and on shortages of supplies for approved swab types.

### SUMMARY

In accordance with the purposes of the present invention, as embodied and broadly described herein, an embodiment of the method using RT-LAMP amplification for detecting nucleic acid from SARS-CoV-2, hereof, includes the steps of: collecting a sample of raw saliva; providing a chemical stabilizer, wherein the saliva is mixed with the chemical stabilizer, forming stabilized saliva; heating the stabilized saliva for a chosen time at a chosen temperature to inactivate the chemical stabilizer, forming heat-treated, stabilized saliva; pre-annealing a first oligonucleotide primer selected from the primers FIP, BIP, F3, B3, Loop F, and Loop B for hybridizing SARS-CoV-2 N gene nucleic acid sequence, the selected first oligonucleotide primer being conjugated to a first fluorophore at its 5′ end, with a first fluorescence quencher for the first fluorophore conjugated to the 3′ end of a first reverse complementary oligonucleotide sequence to the selected first oligonucleotide primer, forming an annealed first primer with a first reverse complementary oligonucleotide; preparing an aqueous solution comprising: DNA polymerase, reverse transcriptase, deoxyadenosine triphosphate, deoxycytidine triphosphate, deoxyguanosine triphosphate, deoxythymidine triphosphate, the primers FIP, BIP, F3, B3, Loop F, and Loop B for hybridizing to the SARS-CoV-2 N gene nucleic acid sequence that were not selected in said pre-annealing step for the first oligonucleotide primer; the annealed first primer with a first reverse complementary oligonucleotide from said pre-annealing step for said first primer; magnesium sulfate, betaine, Tris(hydroxymethyl)aminomethane hydrochloride, ammonium sulfate, potassium chloride, and Tween 20; adding the heat-treated, stabilized saliva to the aqueous solution, forming an amplification solution; heating the amplification solution for a chosen time at a single chosen temperature, whereby the first primer is separated from the annealed first oligonucleotide primer with a first reverse complementary oligonucleotide from the pre-annealing step for the first primer such that said RT-LAMP amplification reaction takes place; cooling the amplification solution following said RT-LAMP amplification reaction for a chosen period of time at a chosen temperature, forming a cooled amplification solution, whereby unused first reverse complementary oligonucleotides are again annealed to unused first oligonucleotide primers; providing an ultraviolet light, wherein the cooled amplification solution is illuminated using the ultraviolet light, and wherein the ultraviolet light has a chosen wavelength such that first oligonucleotide primers incorporated into products formed in the RT-LAMP amplification reaction emit fluorescence radiation; and observing the fluorescence radiation.

In another aspect of the present invention and in accordance with its purposes, as embodied and broadly described herein, an embodiment of the method using RT-LAMP amplification for detecting nucleic acid from SARS-CoV-2, hereof, includes the steps of: collecting a sample of raw saliva; providing a chemical stabilizer, wherein the sample is mixed with the chemical stabilizer, forming stabilized saliva; heating the stabilized saliva for a chosen time at a chosen temperature to inactivate the chemical stabilizer, forming heat-treated, stabilized saliva; pre-annealing a first FIP oligonucleotide primer for hybridizing SARS-CoV-2 N gene nucleic acid having SEQ ID NO: 1, the first FIP oligonucleotide primer being conjugated to a first fluorophore at its 5′ end, with a first fluorescence quencher for the first fluorophore conjugated to the 3′ end of a first reverse complementary oligonucleotide sequence to the first. FIP oligonucleotide primer having SEQ ID NO: 13, forming an annealed first FIP primer with a first reverse complementary oligonucleotide; and pre-annealing a second FIP oligonucleotide primer for hybridizing human RNase P POP7 gene nucleic acid having SEQ ID NO: 7, the second oligonucleotide primer being conjugated to a second fluorophore at its 5′ end, with a second fluorescence quencher for the second fluorophore conjugated to the 3′ end of a second reverse complimentary oligonucleotide sequence to the second FIP oligonucleotide primer having SEQ ID NO: 14, forming an annealed second FIP primer with a second reverse complementary oligonucleotide; preparing an aqueous solution comprising: DNA polymerase, reverse transcriptase, deoxyadenosine triphosphate, deoxycytidine triphosphate, deoxyguanosine triphosphate, deoxythymidine triphosphate, BIP, F3, B3, Loop F, and Loop B first oligonucleotide primers for hybridizing with the SARS-CoV-2 N gene nucleic acid sequence, BIP, F3, B3, Loop F, and Loop B second oligonucleotide primers for hybridizing with the human RNase P POP7 gene nucleic acid control, the annealed first FIP primer with the first reverse complementary oligonucleotide from the pre-annealing step for the first FIP primer, the annealed second FIP primer with the second reverse complementary oligonucleotide from said pre-annealing step for the second HP primer, magnesium sulfate, betaine, Tris(hydroxymethyl)aminomethane hydrochloride, ammonium sulfate, potassium chloride, and Tween 20; adding the heat-treated, stabilized saliva to the aqueous solution, forming an amplification solution; heating the amplification solution for a chosen time at a single chosen temperature, whereby the first FIR primers are separated from the annealed first FIP primers with the first reverse complementary oligonucleotide from the pre-annealing step forthe first FIP primers, and whereby the second FIP primers are separated from the annealed second FIP primers with the second reverse complementary oligonucleotides from the pre-annealing step for the second FIP primers, such that said RT-LAMP amplification reaction takes place; cooling the amplification solution following said RT-LAMP amplification reaction for a chosen period of time at a chosen temperature, forming a cooled amplification solution, whereby unused first reverse complementary oligonucleotides are again annealed to unreacted first FIP primers, and unused second reverse complementary oligonucleotides are again annealed to unreacted second FIP primers; providing an ultraviolet light, wherein the cooled amplification solution is illuminated using the ultraviolet light, and wherein the ultraviolet light has a chosen wavelength such that incorporated fluorophore-conjugated oligonucleotides from said RT-LAMP amplification reaction emit fluorescence radiation; and observing the fluorescence radiation.

Benefits and advantages of the present invention include, but are not limited to, providing a molecular analysis for the detection of pathogen infections, including SARS-CoV-2/COVID-19 infections, that: (1) produces a low rate of false positives or false negatives, through the use of fluorophore oligonucleotides in a two-color combination for visualizing clear, non-ambiguous color differences that indicate the presence or absence of pathogen genetic material, and quencher oligonucleotides for both pathogen and human genetic material that specifically permit amplification and visualization only when accurately detecting the target nucleic acid, thus reducing false positive analyses and false or ambiguous interpretation of resUlts; (2) generates results within hours; (3) does not require expensive laboratory equipment; (4) does not require highly-skilled technicians or medical professionals to perform the analysis; (5) is non-invasive, using a self-collected small volume of treated saliva as a biological sample for the reaction, rather than purified nucleic acid, thereby simplifying and streamlining the molecular analysis over methods that require prior DNA or RNA isolation and purification of DNA or RNA; (6) is not reliant on supply shortages that may exist for swabs since no swabs of any type are required, PPE (personal protective equipment), collection devices, except for a tube having a piece of paper containing a lysis or stabilizing solution, viral transport media, or nucleic acid extraction reagents; and (7) uses a single reaction tube to analyze both sample quality and pathogen genetic material with a clear two-color read-out, which simplifies sample tracking and interpretation over tests that require multiple tubes per sample.

### BRIEF DESCRIPTION OF THE DRAWINGS

The accompanying drawings, which are incorporated in and form a part of the specification, illustrate the embodiments of the present invention and, together with the description, serve to explain the principles of the invention. In the drawings:

FIG. 1 is a flow diagram of an embodiment of the present method.

FIG. 2A illustrates the hybridization of the reverse complementary oligonucleotide having SEQ ID NO: 13 conjugated to the BHQ2 quencher, with the FIP primer having SEQ ID NO: 1 conjugated to TexasRed fluorophore, showing the mismatch marked by the arrow, of a T (thymine) nucleotide bound to a G (guanine) nucleotide instead of an A (adenine) nucleotide, while FIG. 2B illustrates the hybridization of the reverse complementary oligonucleotide having SEQ ID NO: 14 conjugated to the BHQ1 quencher, with the FIP primer having SEQ ID NO: 7 conjugated to FAM fluorophore.

### BRIEF DESCRIPTION OF THE SEQUENCE LISTINGS

SEQ ID NO: 1 discloses the nucleic acid sequence for the SARS-CoV-2 N-gene Forward Internal Primer, FIP.
SEQ ID NO: 2 discloses the nucleic acid sequence for the SARS-CoV-2 N-gene Backward Internal Primer, BIP.
SEQ ID NO: 3 discloses the nucleic acid sequence of the SARS-CoV-2 N-gene Forward External Primer, F3.
SEQ ID NO: 4 discloses the nucleic acid sequence of the SARS-CoV-2 N-gene Backward External Primer, B3.
SEQ ID NO: 5 discloses the nucleic acid sequence of the SARS-CoV-2 N-gene Forward Loop Primer, Loop F.
SEQ ID NO: 6 discloses the nucleic acid sequence of the SARS-CoV-2 N-gene Backward Loop Primer, Loop B.
SEQ ID NO: 7 discloses the nucleic acid sequence of the Human RNaSe P, Forward Internal Primer, FIP.
SEQ ID NO: 8 discloses the nucleic acid sequence of the Human RNase P Backward Internal Primer, BIP.
SEQ ID NO: 9 discloses the nucleic acid sequence for the Human RNase P Forward External Primer, F3.
SEQ ID NO: 10 discloses the nucleic acid sequence of the Human RNase P Backward External Primer, B3.
SEQ ID NO: 11 discloses the nucleic acid sequence for the Human RNase P Forward Loop Primer, Loop F.
SEQ ID NO: 12 discloses the nucleic acid sequence for the Human RNase P Backward Loop Primer, Loop B.
SEQ ID NO: 13 discloses the nucleic acid sequence for the SARS-CoV-2 N-gene quencher probe.
SEQ ID NO: 14 discloses the nucleic acid sequence for the Human RNase P quencher probe.

### DETAILED DESCRIPTION

It is desirable to have a process for detection of target genetic material that uses low-complexity, non-invasive biological sample collection techniques, that is rapid, easy-to-use, highly accurate, does not require specialized laboratory equipment, as most current viral molecular tests rely on expensive temperature-cycling machines that are not available in most point-of-care settings, and that does not require significant specialized training to collect a sample, which can be self-collected through salivating or drooling into a test tube, perform the reaction, or interpret the results. All target nucleic acids sequences have been chosen to provide a high level of accuracy and sensitivity. Embodiments of the present method combine reverse transcription, loop-mediated isothermal amplification (RT-LAMP) technology with oligonucleotide primers, fluorophore-labeled oligonucleotides, quencher technology, buffer components, enzymes, and enzyme ratios, chosen to minimize the false positive and false negative results that often accompany the use of RT-LAMP, The present method includes internal positive control targeting sequences, allowing significant confidence in interpretation of results, can be performed at a single elevated temperature, thus eliminating the need for specialized laboratory equipment, and can be completed in 1-2 h, allowing results to be returned in a rapid manner. Further, the results of the reactions can readily be interpreted by personnel without significant specialized training by observing the fluorescence color of the reaction using ultraviolet light.

Loop-mediated isothermal amplification, LAMP, technology has been used to detect pathogens, such as malaria and salmonella, as examples. LAMP merged with reverse transcriptase, RT-LAMP, has been used to detect viral RNA in HIV and several respiratory RNA viruses, including SARS-CoV-2. See, e.g., “A Molecular Test Based On RT-LAMP For Rapid, Sensitive and Inexpensive Colorimetric Detection of SARS-CoV-2 In Clinical Samples” by Catarina Amaral et al., in Scientific Reports 11, Article Number 16430 (2021), where COVID-19 was detected using RNA extraction-tree RT-LAMP from self-collected saliva. Hairpin-forming LAMP primers first invade the DNA template, which is then annealed and extended as catalyzed by a strand-displacing DNA polymerase. In the initiation of amplification, the annealed primers are used to prime the action of a strand displacement enzyme, leading to the formation of a dumbbell-like single-strand DNA loops, which form the basis for amplification and elongation. Forward and backward inner LAMP primers hybridize to the complementary and reverse complimentary target sequences. The product of LAMP is a series of concatemers of the target region.

Because LAMP uses 4-6 primers targeting 6-8 regions within a small segment of the genome, which primers have many constraints, commercial software is often used to assist with primer design. The large number of primers per target increase the likelihood of primer-primer interaction, and the incidence of false positive and false negative results. See, “Reduced False Positives and Improved Reporting of Loop-Mediated Isothermal Amplification Using Quenched Fluorescent Primers” by Patrick Hardinge and James A. H. Murray, in Scientific Reports 9, Article Number 7400 (2019). As will be discussed in detail below, careful primer design and optimal reaction conditions and testing are used to minimize these problems.

Briefly, embodiments of the present invention include: a method for rapidly and accurately detecting target nucleic acid, having the following steps: (1) collecting a biological sample; (2) transferring a small amount of the sample to an optically clear reaction test tube containing: (a) a reverse transcriptase (an enzyme used to generate complementary DNA (cDNA) from an RNA template); (b) deoxyribonucleotide triphosphates (dNTPs) (the building blocks of DNA, which lose two of phosphate groups when incorporated into DNA during replication); (c) a strand-displacement DNA polymerase (an enzyme that catalyzes the synthesis of DNA from nucleoside triphosphates, by adding nucleotides to the (3′)-end of a DNA strand, one nucleotide at a time; (d) oligonucleotide primers specifically contacting the target nucleic acid sequences of either the pathogen nucleic acid or the human control gene nucleic acid; (e) fluorophore-conjugated oligonucleotide primers for specifically contacting the target nucleic acid sequences of either the pathogen nucleic acid or the human control gene nucleic acid; and (f) quencher-conjugated oligonucleotides specifically contacting the fluorophore-conjugated oligonucleotide primers, the requisite materials for a reverse transcription loop-mediated isothermal amplification reaction (RT-LAMP) (It should be mentioned that the reactions may be performed in optically clear microplates containing a chosen number of individual wells); (3) incubating the reaction under conditions such that cDNA synthesis and isothermal amplification takes place, thereby generating DNA amplification products; (4) cooling the resulting reaction products under conditions permissive for oligonucleotide hybridization thereby quenching unincorporated fluorophore-conjugated oligonucleotide primers; (5) detecting specific signals for incorporated fluorophore-conjugated oligonucleotide primers, wherein detection of one fluorophore signal indicates the presence of the human control gene nucleic acid and correct functioning of the reaction, and detection of the other fluorophore signal indicates the presence of the pathogen nucleic acid; and (6) interpreting the results, wherein detection of either or both fluorophore signal combinations indicates the presence of a quantity of the target nucleic acids present in the sample, while failure to detect the pathogen fluorophore signal indicates the absence of target nucleic acid in the sample, and failure to detect any fluorophore signal indicates that the reaction has failed.

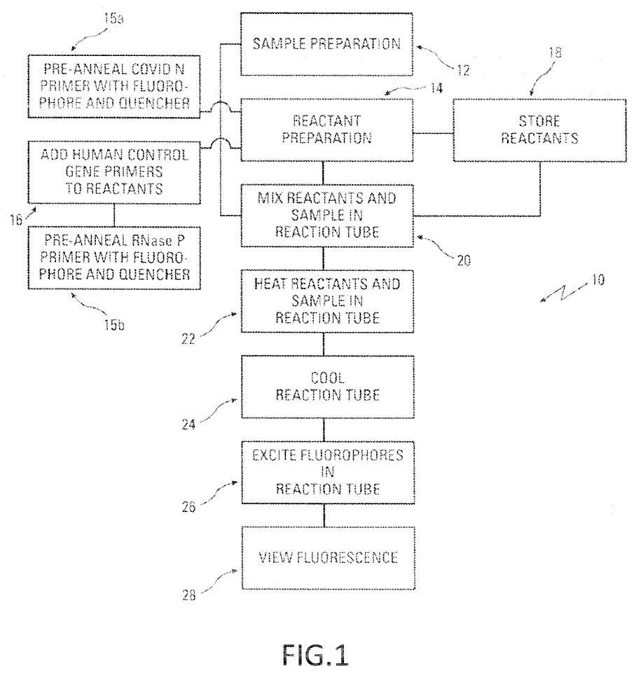

Reference will now be made in detail to the present embodiments of the invention, examples of which are illustrated in the accompanying drawings. It will be understood that the FIGURES are presented for the purpose of describing particular embodiments of the invention and are not intended to limit the invention thereto. Turning now to FIG. 1, represented is a flow diagram, 10, of an embodiment of the present method. In step 12, the biological sample for analysis is collected by depositing 1 mL of passive saliva obtained from drooling or spitting into a 5 mL test tube containing powdered proteinase K and a thin barrier. The barrier can be pushed aside using a small plastic rod, allowing the saliva to mix with the stabilization proteinase, or the barrier has holes that permit the saliva to reach the bottom without needing to use the plastic rod. Other collection tubes do not contain a barrier, and the saliva will move to the bottom of the tube where it can come in contact with powdered proteinase K. The test tube is then capped and incubated at ambient temperature for up to 48 h, and typically greater than 15 min., to permit the proteinase K to react, after which it is heat-treated at greater than or equal to about 95° C. for 10 min. to inactivate the proteinase K, which would otherwise destroy the assay enzymes. The sample is then allowed to cool, and can be stored. It should be mentioned that there is no RNA isolation for either the SARS-CoV-2 or the human templates in the clinical samples employed, since the SARS-CoV-2 is an RNA pathogen, and the human gene is encoded in cellular DNA for all humans, but expressed/transcribed into RNA which is present in the saliva. The proteinase K lyses the cells in the saliva sample, releasing additional RNA. The biological sample may include buffered saliva; materials collected using a nasal swab, nasopharyngeal swab, or throat swab; RNA isolated from untreated saliva; RNA isolated from buffered saliva; RNA isolated from material collected using a nasal swab, nasopharyngeal swab, or a throat swab; DNA isolated from untreated saliva; DNA isolated from buffered saliva; DNA isolated from material collected using a nasal swab, nasopharyngeal swab, or a throat swab. Stabilization chemicals may also include one or a combination of sodium dodecyl sulfate, sodium lauryl sulfate, guanidinium thiocyanate, guanidine hydrochloride, styrene divinylbenzene copolymer, polyethylene glycol, and/or Chelex.

As will be set forth in greater detail below, Step 14, reactant preparation, includes mixing of Bst 2.0 NON-WarmStart DNA polymerase, NEB WarmStart RTX reverse transcriptase (the reverse transcriptase enzyme and the strand-displacement DNA polymerase may be the same or different enzymes), Antarctic thermolabile uracil DNA glycosylase, deoxyadenosine triphosphate, deoxycytidine triphosphate, deoxyguanosine triphosphate, deoxythymidine triphosphate, deoxyuridine triphosphate, magnesium sulfate, betaine, tris hydrochloride, ammonium sulfate, potassium chloride, Tween 20, water, 5 oligonucleotide primers that specifically bind or hybridize to the target SARS-CoV-2 N gene nucleic acid, and, as will be described in more detail below, a sixth oligonucleotide primer conjugated to a chosen fluorophore and pre-annealed to a reverse complementary oligonucleotide conjugated to a quencher for the chosen fluorophore, for a total of 20 μL in an optically clear 0.2 mL reaction tube. It should be mentioned that the six oligonucleotide primers bind to the SARS-CoV-2 N gene nucleic acid, and take part in the RT-LAMP amplification process.

Step 15a of FIG. 1 illustrates one of the two pre-annealing steps. The FIP primer having SEQ ID NO: 1 was chosen for conjugation to a, TexasRed or another fluorophore. It should be mentioned that one of the other oligonucleotide primers, BIP, F3, B3, Loop F, and Loop B, could have been selected in place of the FIP primer. The quencher used for targeting the TexasRed fluorophore is BHQ2 and the oligonucleotide sequence having SEQ ID NO: 13 is reverse complementary to the oligonucleotide conjugated to the TexasRed fluorophore. The primer and quencher were supplied in two separate tubes as lyophilized components from a supplier thereof, wherein the fluorophore and the quencher are conjugated to their respective oligonucleotides as purchased, and are then independently dissolved in water to a concentration of 100 μM. Primer and quencher aqueous solutions were mixed to together at an experimentally determined ratio ranging from between 1:1 and 1:3 of primer to quencher, depending on the affinity and potential hybridization of the quencher to the template. This mixture was heated to about 85° C. for about 2 min. to remove any secondary structure of the oligonucleotides, and slowly cooled to room temperature over an about 5 min. interval. Slow cooling allows the quencher to bind or anneal to the primer. The resulting pre-annealed solution was then added to the reaction mixture in the appropriate concentration, as part of Step 14, which increases the stability of the premixed reagents.

Step 15b illustrates the pre-annealing step for the human RNase P FIP primer and quencher described in Step 16 in a different tube, and was identically performed separately from the pre-annealing of the FIP/Quencher combination for the COVID N-gene FIP and quencher set forth above. These separately pre-annealed combinations are added to the reaction solution, as appropriate, while the other primers are added directly to the reaction solution. The FIP primer having SEQ ID NO: 7 was chosen for conjugation to a FAM or another fluorophore, and the oligonucleotide sequence having SEQ ID NO: 14 and conjugated to a BHQ1 quencher, is reverse complementary to the oligonucleotide conjugated to the FAM fluorophore. It should be mentioned that one of the other oligonucleotide primers, BIP, F3, B3, Loop F, and Loop B, could hav/e been selected in place of the FIP primer.

Step 16 shows the optional mixing of 5 additional cligonucleotide primers known for hybridizing with the human RNase P POP7 gene nucleic acid sequence, used here as a control target gene, along with one of the primers being conjugated to a FAM fluorophore. It should be mentioned that any target human control gene nucleic acid molecule can be utilized. The quencher targeting the FAM fluorophore is BHQ1 and the oligonucleotide sequence is reverse complementary to the oligonucleotide conjugated to the FAM fluorophore. As will be described below, the RNaseP POP7 gene nucleic acid control may be added to the reactants to verify that the reactions properly occurred, and to minimize false negatives. Step 18 shows that the mixed reactants may be stored for several weeks if cooled to between about 4° C. and about −20° C., and retain its viability.

In Step 20, 5 μL of the biological sample (from the 5 mL test tube) from Step 12 is transferred to a 0.2 mL test tube containing 20 μL of reactants from Step 14 using a micropipette, an absorbent dipstick, a bulb pipette, a dropper, a capillary tube, or a loop transfer tool. It has been found that between about 2 μL and about 10 μL is an adequate sample size. In Step 22, the reaction is incubated using a dry heat block for about 45 min. (between about 30 min. and about 80 min. has been found to be adequate) at about 65° C. (between about 55° C. and about 70° C.), after which the reaction is cooled to about 21° C. for about 5 min., as seen in Step 24, such that unincorporated fluorophore-conjugated oligonucleotides are re-annealed to complementary quencher oligonucleotides and no longer can produce visible fluorescence. It should be mentioned that any heating device capable of maintaining the reaction at the chosen constant temperature, such as a water bath, PCR apparatus, as examples, can be used. Fluorophore-conjugated oligonucleotides that have already been incorporated into an amplicon will not be available to re-anneal to complementary quencher oligonucleotides, and will produce visible fluorescence under ultraviolet light. In Step 26, reactions are viewed with an ultraviolet transilluminator viewing device (having a uv filter to protect a viewer's eyes), and fluorescence is interpreted as positive for a viral pathogen or other target signal if the reaction glows in a spectrum from red to orange to yellow, as positive for the human signal and negative for viral pathogen signals if the reaction glows green, and with no colored fluorescence in a reaction interpreted as a failed reaction. The fluorophores and the ratios of primer sets were chosen such that the green (human) fluorescence does not overpowers the red (viral). Therefore, if there are detectable levels of viral target, the combination of both fluorescences will either show fully red (for viral), or a red spectrum (for the combination), but not fully green. In use, about 2.5 times as much of the virus primer set as the human primer set was found to achieve this effect. Examples of observable fluorescence (or lack thereof) may be made available to a user by supplying (a) Positive control reactions containing both human and SARS-CoV2-RNA; (b) Negative control reactions only containing human RNA; and (c) Reactions containing no RNA, with each set of reaction tubes in a kit, as will be described in more detail below.

In Step 28, fluorescence may be documented by a digital photograph, a camera, light box, or electronic image acquisition system.

Having generally described embodiments of the invention, the following EXAMPLES demonstrate further aspects thereof.

### Example 1

### A. Selection of Primers for Step 14

### 1. Primers Specific to SARS-CoV-2 and Control Nucleic Acid

Commercially available primers that will amplify the selected target regions were used to detect the SARS-CoV-2 genome (the complete genome for the Wuhan-Hu-1 isolate having GenBank: MN908947.3, with the N nucleocapsid phosphoprotein having Gene ID: 43740575), and the RNase P POP7 human control gene (NCBI Entrez Gene: 10248, Ensembl ID: ENSG00000172336), in the sample. These primer sequences were chosen since they are predicted to specifically amplify target regions without off-target interactions, and/or lack of negative interactions with either other included primers or other biological sample genetic material. The primers utilized for the RTLAMP amplification, along with two quencher probes SEQ ID NO: 13 and SEQ ID NO: 14 are set forth in the TABLE.

TABLE

| PRIMER NAME | TARGET | SEQUENCE |
|---|---|---|
| SARS-CoV-2 N-gene | SARS-CoV-2 N | SEQ ID NO: 1 |
| Forward Internal Primer, | gene | /TexasRed/TGCGGCCAATGTTTGT |
| FIP | | AATCAGCCAAGGAAATTTTGGGGAC |
| SARS-CoV-2 N-gene | SARS-CoV-2 N | SEQ ID NO: 2 |
| Backward Internal Primer, | gene | CGCATTGGCATGGAAGTCACTTTGAT |
| BIP | | GGCACCTGTGTAG |
| SARS-CoV-2 N-gene | SARS-CoV-2 N | SEQ ID NO: 3 |
| Forward External Primer, | gene | AACACAAGCTTTCGGCAG |
| F3 | | |
| SARS-CoV-2 N-gene | SARS-CoV-2 N | SEQ ID NO: 4 |
| Backward External Primer, | gene | GAAATTTGGATCTTTGTCATCC |
| B3 | | |
| SARS-CoV-2 N-gene | SARS-CoV-2 N | SEQ ID NO: 5 |
| Forward Loop Primer, | gene | TTCCTTGTCTGATTAGTTC |
| LoopF | | |
| SARS-CoV-2 N-gene | SARS-CoV-2 N | SEQ ID NO: 6 |
| Backward Loop Primer, | gene | ACCTTCGGGAACGTGGTT |
| LoopB | | |
| SARS-CoV-2 N-gene | SARS-CoV-2 N- | SEQ ID NO: 13 |
| quencher probe | gene FIP | ATTGGTCGCA/BHQ2/ |
| Human RNaseP, Forward | RNaseP-POP7 | SEQ ID NO: 7 |
| Internal Primer, FIP | | /FAM/TGGACCCTGAAGA |
| | | CTCGGTTTTAGCCACTGA CTC |
| | | GGATC |
| Human RNaseP Backward | RNaseP-POP7 | SEQ ID NO: 8 |
| Internal Primer, BIP | | CCTCCGTGATATGGCTCTTCGTTT |
| | | TTTTCTTACATGGCTCTGGTC |
| Human RNaseP Forward | RNaseP-POP7 | SEQ ID NO: 9 |
| External Primer, F3 | | TTGATGAGCTGGAGCCA |
| Human RNaseP Backward | RNaseP-POP7 | SEQ ID NO: 10 |
| External Primer, B3 | | CACCCTCAATGCAGAGTC |
| Human RNaseP Forward | RNaseP-POP7 | SEQ ID NO: 11 |
| Loop Primer, LoopF | | ATGTGGATGGCTGAGTTGTT |
| Human RNaseP Backward | RNaseP-POP7 | SEQ ID NO: 12 |
| Loop Primer, LoopB | | CATGCTGAGTACTGGACCTC |
| Human RNaseP quencher | Human RNaseP | SEQ ID NO: 14 |
| probe | FIP | TCAGGGTCACA/BHQ1/ |

Note that lowaBlack FQ (IDT) is another dark quencher that can be used in place of BHQ1 to quench the FAM fluorescence, and lowaBlack-RQ (IDT) can be used in place of BHQ2 to quench the TexasRed fluorescence.

### B. Selection of Fluorophores for Step 14

To allow efficient amplification of target sequences, while enabling visualization, oligomeric nucleotide conjugated fluorophores (bound to the Forward Internal Primer, FIP, set forth above) were selected to allow visualization by eye using a single light source. This is accomplished by utilizing two individual fluorophores that can each be excited with a single UV light source, but that emit two distinct wavelengths and can be simultaneously seen by eye. Fluorophores are also selected such that they do not inhibit or hinder sequence amplification from the specific targets of interest. Fluorophore combinations may consist of any commercially, available visible conjugated (chemically bonded, conjugated, or attached to the oligonucleotide during synthesis) fluorophore. The number of selected fluorophores can range from one to many (>5). A single fluorophore or multiple fluorophores can be used to target multiple pathogens or multiple sequences in a single pathogen, from the same assay. The assay may or may not contain a separate fluorophore that targets a control host gene/transcript. Wavelength filters may be employed, whereby the emission from 1 fluorophore is observed at a time. Any host gene DNA or RNA may be used as a target for the internal control. Human RNaseP gene was selected because it is well-expressed and is a commonly used human control gene.

### C. Selection of Quenchers for Step 14

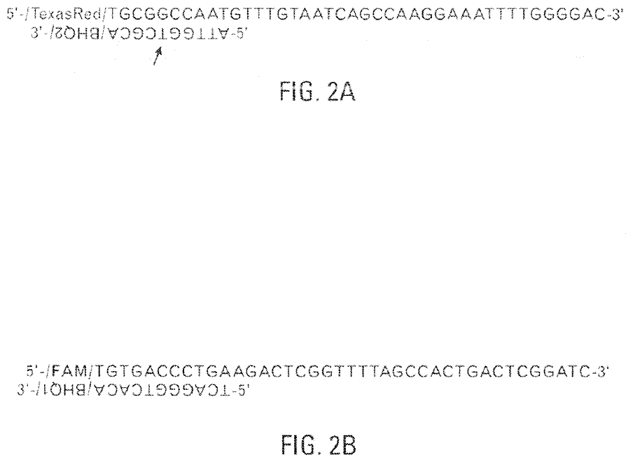

To quench fluorescence from unincorporated primers, quencher oligomers are selected to contain the appropriate conjugated molecule effective for quenching the specific wavelength of fluorescence emitted from the fluorophores used in the assay. Quencher oligonucleotide sequences are chosen to have a specific length, and may contain mismatches, which allow binding of the quencher oligomer to a particular fluorescently labelled oligomer at low temperatures (<50° C.), but not at high temperature (>50° C.). FIG. 2A illustrates the hybridization of the reverse complementary oligonucleotide having SEQ ID NO: 13 conjugated to the BHQ2 quencher, with the FIP primer having SEQ ID NO: 1 conjugated to TexasRed fluorophore showing the mismatch marked by the arrow of a T (thymine) nucleotide bound to a G (guanine) nucleotide instead of an A (adenine) nucleotide. FIG. 2B illustrates the hybridization of the reverse complementary oligonucleotide having SEQ ID NO: 14 conjugated to the BHQ1 quencher, with the FIP primer having SEQ ID NO: 7 conjugated to FAM fluorophore. When reverse complementary sequences hybridize, “mismatches” mean any non-Watson-Crick base-pairing pairs. Mismatches alter the hybridization strength of the duplex, making it weaker, and, along with the chosen length of the quencher, produce the effect of temperature on binding. That is, binding of the quencher to its target fluorophore becomes possible at low temperatures, while the two oligonucleotides separate at higher temperatures, such that at the assay temperature, RT-LAMP amplification can occur, Online programs such as those available on the websites of Sigma or IDT were used to predict suitable quenchers based on sequence and calculated hybridization strength, delta G, but their actual operation must be tested empirically. Therefore, quencher oligonucleotides sequences are selected such that they do not inhibit amplification of either the target pathogen or the host control transcript/gene. Quencher conjugated oligonucleotides were purchased from commercial sources, with and without mismatches, and with a variety of lengths, in order to optimize their properties.

Other quenchers that may be used include: (a) lowaBlack-FQ, which can quench the FAM fluorophore in the human target; (b) lowaBlack-RQ, which can quench the TexasRed fluorophore in the COVID target; (c) TAMRA, which can quench the FAM fluorophore; and (d) BlackBerry Quencher 650, which can quench the TexasRed fluorophore, as examples. The FAM and TexasRed fluorophores, and the above quenchers conjugated to oligonucleotides are commercially available from multiple companies including: IDT, GeneWiz, ThermoFisher, Abcaen, and Biosynthesis, as examples.

### Example 2

### A. Optimization of Primers for Step 14

The ratio of different primer sets targeting multiple sequences for allowing amplification of all targets, and for facilitating visualization of all amplified targets, was determined experimentally. RTLAMP primer sets consist of 6 oligomers for targeting each region of interest. An advantageous ratio between these sets was determined experimentally by using a range of ratios, for example, 1:0.5, 1:1, 1:1.5, 1:2, etc. The ratio that gave efficient amplification from all targets and allowed visual analysis of the results was found to be 1:2.5 of human primers to viral primers. Considerations for this process included: transcription level of pathogen(s) target verses control target, expected pathogen load in the sample compared to the target, brightness of selected fluorophores, ease of visualization of different wavelengths by the human eye (i.e, green is more readily seen than red), and the number of different sequences targeted by the same fluorophore in the assay. This process may include other considerations or ratios that are not listed depending on the specific requirements and biology of the organisms targeted.

### B. Optimization of Quenchers for Step 14

Primer and quencher ratios were optimized to allow simultaneous amplification and visualization of the targets in the assay. Various ratios of fluorophore conjugated primer and quencher were evaluated (i.e., 1:1, 1:1.5, 1:3, 1:5, and 1:6.5 molar excess of quenchers over fluorophore FIPs) to define a ratio that results in complete quenching of fluorescence from unincorporated fluorescently conjugated oligomers. A 1:1 ratio was found not to be sufficient to completely quench fluorescence when there was no nucleic acid, which would be a false positive. Ratios were also optimized to maintain quenching without inhibiting amplification of any target in the assay.

In an RT-LAMP reaction the first step is conversion of the RNA to complementary DNA (cDNA) carried out by an RT enzyme included in the reaction. The oligomers that prime this process are the same reverse primers that catalyze the loop mediated amplification by the polymerase in the next step. However, the use of quencher technology also means that a reverse complement sequence that can bind the target RNA template itself is introduced. Binding of free quencher to the RNA template prior to heating the reaction can lead to reaction inhibition because the interaction blocks reverse transcription of the sequence needed to initiate amplification. Additionally, free primers can initiate non-specific amplification at low temperatures in all LAMP reactions. Therefore, the reaction components need to be mixed immediately prior to running the reaction, which is what is customarily done. Prehybridization (pre-annealing) steps (heating together and slow-cooling to promote annealing) the fluorophore-conjugated oligomers (SEQ ID NO: 1 and SEQ ID NO: 7) with quencher oligomers (SEQ ID NO: 13 and SEQ ID NO: 14, respectively) were optimized to yield efficient quenching in the absence of targets. That is, temperature and timing for prehybridization were optimized to maintain quenching without inhibiting amplification of the chosen targets (SARS-CoV-2 RNA and human RNaseP transcript) in the assay, thereby reducing false positives and false negatives. By adding the pre-annealing step, the free quencher and primers are “locked” together and resulting duplex becomes “hot-start”. Without this added process the reaction does not amplify. This significantly reduces non-specific amplification allowing the present chemistry to be stable for days at room temperature or longer, if chilled, as a pre-mixed, liquid reaction solution.

Further, if the reaction continues to react after the designated time, the visual readout can change from negative to positive, since the enzymes are still available and there is an increased opportunity that they can locate an off-target species and beginning amplification of that. Thus, if left to react for long periods all such analyses will likely become positive. To reduce this possibility, LAMP enzymes are often inactivated at the end of the designated reaction time by heating the reaction to a high temperature (around 85° C.) for 5-15 minutes, which destroys the enzymes and stops such reactions from progressing. This additional step is inconvenient for the present point-of-care assay, where a thermal cycler is generally not available. Effectively quenching the fluorophore FIP primer once the reaction time is over by slow-cooling to room temp accomplishes this result, and if the enzymes still remain active, a false positive is not observed, since the read-out is fluorescence and that fluorescence is quenched.

### C. Optimization of Buffers for Step 14

Concentrations of buffer and other components for maximizing reaction efficiency and efficacy were:
(a) dNTPs are free deoxy-triphosphates (A, T, C, and G) that serve as the raw materials for amplification. Concentrations can range between 5 mM and 15 mM for each dNTP, dNTPs from any source in either powdered or liquid form can be used.
(b) deoxyuridine triphosphate when used in combination with thermolabile uracil DNA glycosylase is a component that destroys carry-over contamination prior to new amplification, and minimizes false positives. Concentrations can range between about 2.5 mM and about 7.5 mM. This component is optional, but improves performance of the assay and also reduces the risk of cross-contaminating future assays with previously amplified results. Additional methods to reduce non-specific amplification include CRISPR/Cas9 targeted degradation, and use of reaction sinks using a pseudo template. Amplification products from one run of a test contaminating future reactions can lead to false positives and is a problem for any nucleic acid test, including both LAMP and PCR. If a small amount of amplification product contaminates a new test, that test will appear positive because the primers/enzymes will amplify that molecule regardless of the presence or absence of target viral nucleic acid. Including dUTP and thermolabile UDG (uracil DNA glycosylase; NEB catalog #M0372S (Antarctic Thermolabile UDG)), while not necessary for the reaction to proceed, reduces the chance of these false positives, since when included, dUTP is incorporated at some low frequency in the amplification products. If those amplification products accidentally contaminate a new test (as happens regularly), the UDG enzyme in the new test degrades any nucleic acid containing UDG. However, this process only works at room temperature, and once the reaction is heated, the thermolabile UDG is inactivated and does not degrade the new amplicons of the new test.
(c) Magnesium sulfate is necessary for catalytic activity of enzymes in the assay and adjusting its concentration allows increased or decreased reaction temperature. Concentrations may vary between 2 mM and 10 mM.
(d) Betaine is used for altering the hybridization potential of oligomers and templates in the reaction. Concentrations may range between 0 mM and 0.8 mM and are adjusted tor each new assay. Alternatives or additional components include DMSO (dimethyl sulfoxide), proline, trehalose and ionic liquids including imidazolium, pyridinium, pyrrolidinium, and phosphonium, with the anions including halides, tetrafluoroborate (BF₄⁻), hexafluorophosphate (PF₆⁻), and bis[(trifluoromethyl) sulfonyl]imide.
(e) Tris hydrochloride is a buffer that maintains the pH of that solution for allowing optimal activity of the enzymes. Concentrations may vary between 10 mM and 50 mM and the pH may be between 6.5 and 9.
(f) Ammonium sulfate and potassium chloride stabilize the enzymes that catalyze the reaction. Ammonium sulfate concentration may vary between 5 mM and 20 mM and potassium chloride from between 50 mM and 150 mM.
(g) Tween 20 is a polysorbate-type nonionic surfactant, and assists in increasing the specificity of the reaction. Concentrations may vary between 0.05% and 0.3%. Alternatives include Triton X and NP-40.

### D. Additional Components that May be Utilized in Step 14, Depending on the Specifics of Each Assay

(a) Crowding reagents for increasing enzyme specificity and accelerate reaction rate, and include: Polyethylene glycol (PEG), Ficoll, Dextran.
(b) Duplex strengtheners for increasing reaction efficiency for weak primer/template pairs, and include Tetramethylammonium chloride.
(c) Enzyme stabilizers—for increasing enzyme activity, enzyme stabilization, and enzyme protection, which can improve assay robustness and reproducibility, and include bovine serum albumin (BSA), pullulan, trehalose, Proline, glycine in combination with trehalose, betaine, sucrose, maltose, ectoine, hydroxyectoine, sorbitol, glycine betaine and homodeanol betaine.
(d) Template Blocking reagents for decreasing nonspecific interactions between nucleic acids and polymerase or between nucleic acids, protecting nucleic acids from degradation, and decreasing background fluorescent signals. These reagents may improve sensitivity and specificity, and include: single-stranded DNA binding proteins, graphene oxide, Chelex100 resin, and cobalt oxyhydroxide (Co) nanoflakes.

### E. Optimization of Enzymes for Step 14

(a) Strand-displacement polymerase enzyme is an enzyme that catalyzes the amplification of the target DNA using added primer sets. Any strand displacement polymerase may be used including, but not limited to: BST 2.0, BST 2.0-hotstart, BST 3.0 (New England Biolabs), EquiPhi29 and Bsm DNA polymerases (ThermoFisher Scientific), losPol Bst (ArticZyme Technologies), or any in lab produced polymerase enzyme with strand displacement activity. Enzyme concentrations can vary depending on manufacturer defined units and are optimized for each assay. A final reaction concentration of 0.4 units/4 (10 units per 25 μL reaction) of Bst 2.0 DNA Polymerase (NEB catalog #M0537S) and/or Bst 2.0 WarmStart® DNA Polymerase (NEB catalog #M0538S) was employed.
(b) Reverse transcription enzyme is an enzyme that catalyzes the conversion of RNA into complementary DNA, allowing detection of RNA targets (such as RNA viruses and viroids). Any commercial or in lab produced enzyme that converts RNA into complementary DNA may be used in the assay. Concentrations may vary depending on manufacture defined units. This component is optional depending on the nature of the target to be amplified. A final reaction concentration of 0.375 units/4 of WarmStart® RTx Reverse Transcriptase (9.375 units per 25 μL reaction) was employed (NEB catalog #M0380S (WarmStart® RTx Reverse Transcriptase)
(c) Antarctic thermolabile uracil DNA glycosylase is an enzyme that degrades DNA molecules that contain dUTP preventing carry-over contamination from previous reactions, as described above. Any commercially available or lab produced enzyme that cleave DNA at sites of dUTP incorporation can be used in the assay. This component is optional, but improves performance and specificity of the assay, and reduces the risk of cross-contaminating future assays with previously amplified results. A final reaction concentration of 0.025 units/pt (0.625 units per 25 μL reaction) of Antarctic Thermolabile UDG (NEB catalog #M0372S) was employed.

### Example 3

Optimization of Reaction Conditions for Steps 20 and 22:
(a) Reaction temperatures can vary between about 55° C. and about 70° C. and depend on the primer sequences and enzymes selected for the assay. This condition is optimized for each new assay.
(b) Reaction time, or incubation time at the reaction temperature is optimized to allow a specific limit of detection to be achieved. Reaction time can vary between about 15 min. and about 120 min. depending on primer sequences, the pathogen concentration likely to be present in the sample and the brightness of the fluorophores.
(c) Cooling reaction products after the amplification reaction is optimized to allow greatest quenching of non-incorporated fluorescent oligos and fluorescence detection of specific amplification products.

### Example 4

Embodiments of the present method for detecting SARS-CoV-2/COVID-19 nucleic acid may be included in a kit for reverse transcription loop-mediated isothermal amplification and fluorescent detection of the pathogen nucleic acid, along with a human control gene, from biological samples, including raw saliva, using the specific oligonucleotide primers, fluorophore-labeled probes, buffers, enzymes, and quenchers identified above. Collection tubes, an optional rod, and instructions are packaged in individual plastic bags for sample self-collection. Other collection kit formats could be used in other versions.

A kit may include: (A) at least one 5 mL volume screw-capped saliva sample tubes having a dried stabilization component, a thin barrier, and a sticker label with an identifier code. A small plastic rod used to push aside the thin barrier after saliva is in the tube; (B) At least one fixed-volume micropipette, or a fixed-volume capillary tube having a plunger, capable of transferring a 5 μL sample volume and associated pipette tips; (C) at least one optically-clear 0.2 mL volume reaction tubes that may be attached in groups of 8 tubes, each tube having individual attached snap cap, being capable of withstanding the elevated temperature of about 65° C. and remain sealed, and containing a chosen volume of the aqueous solution; (D) a dry heating block or water bath capable of being heated for heating the reaction tubes to about 65° C. and the saliva sample tubes to >95° C.; (E) an ultraviolet lamp; (F) an ultraviolet filter for viewing fluorescence, and (G) a set of 3 control reactions for visual comparison of fluorescence results, as described above, including (a) a positive control reaction containing both human and SARS-CoV-2 RNA; (b) a negative control reaction containing only human RNA; and (c) a no-reaction control containing no RNA. A dual dry heating block for holding the 0.2 mL tubes on one side and the 5 mL tubes on the other side has been used.

The foregoing description of the invention has been presented for purposes of illustration and description and is not intended to be exhaustive or to limit the invention to the precise form disclosed, and obviously many modifications and variations are possible in light of the above teaching. The embodiments were chosen and described in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

What is claimed is:

1. A method using RT-LAMP amplification for detecting nucleic acid from SARS-CoV-2, comprising the steps of:
collecting a sample of raw saliva;
providing a chemical stabilizer, wherein the saliva is mixed with the chemical stabilizer, forming stabilized saliva;
heating the stabilized saliva for a chosen time at a chosen temperature to inactivate the chemical stabilizer, forming heat-treated, stabilized saliva;
pre-annealing a first oligonucleotide primer selected from the primers FIP, BIP, F3, B3, Loop F, and Loop B for hybridizing SARS-CoV-2 N gene nucleic acid sequence, the selected first oligonucleotide primer being conjugated to a first fluorophore at its 5′ end, with a first fluorescence quencher for the first fluorophore conjugated to the 3′ end of a first reverse complementary oligonucleotide sequence to the selected first oligonucleotide primer, forming an annealed first primer with a first reverse complementary oligonucleotide;
preparing an aqueous solution comprising: DNA polymerase, reverse transcriptase, deoxyadenosine triphosphate, deoxycytidine triphosphate, deoxyguanosine triphosphate, deoxythymidine triphosphate, the primers FIP, BIP, F3, B3, Loop F, and Loop B for hybridizing the SARS-CoV-2 N gene nucleic acid sequence that were not selected in said pre-annealing step for said first primer; the annealed first primer with a first reverse complementary oligonucleotide from said pre-annealing step for said first primer; magnesium sulfate, betaine, Tris(hydroxymethyl)aminomethane hydrochloride, ammonium sulfate, potassium chloride, and Tween 20;
adding the heat-treated, stabilized saliva to the aqueous solution, forming an amplification solution;
heating the amplification solution for a chosen time at a single chosen temperature, whereby the first primer is separated from the annealed first oligonucleotide primer with a first reverse complementary oligonucleotide from said pre-annealing step for the first primer such that said RT-LAMP amplification reaction takes place;
cooling the amplification solution following said RT-LAMP amplification reaction for a chosen period of time at a chosen temperature, forming a cooled amplification solution, whereby unreacted first reverse complementary oligonucleotides are again annealed to unreacted first oligonucleotide primers;
providing an ultraviolet light, wherein the cooled amplification solution is illuminated using the ultraviolet light, and wherein the ultraviolet light has a chosen wavelength such that first primers incorporated into products formed in said RT-LAMP amplification reaction emit fluorescence radiation; and
observing the fluorescence radiation.

2. The method of claim 1, wherein the first primer comprises the FIP for the SARS-CoV-2 N gene nucleic acid sequence.

3. The method of claim 2, wherein the first primer has a sequence of SEQ ID NO: 1, and the first reverse complimentary oligonucleotide has a sequence of SEQ ID NO: 13.

4. The method of claim 1, wherein the first fluorophore comprises Texas Red, and the first fluorescence quencher is chosen from BHQ2, lowaBlack-RQ, and BlackBerry Quencher 650.

5. The method of claim 1, further comprising the step of pre-annealing a second oligonucleotide primer selected from the primers FIP, BIP, F3, B3, Loop F, and Loop B for hybridizing human RNase P POP7 gene nucleic acid sequence, the second selected oligonucleotide primer being conjugated to a second fluorophore at its 5′ end, with a second fluorescence quencher for the second fluorophore conjugated to the 3′ end of a reverse complimentary second oligonucleotide sequence to the selected second oligonucleotide primer, forming an annealed second oligonucleotide primer with a second reverse complementary oligonucleotide.

6. The method of claim 5, wherein the aqueous solution further comprises FIP, BIP, F3, B3, Loop F, and Loop B second oligonucleotide primers for hybridizing the human RNase P POP7 gene nucleic acid control molecule that were not selected in said pre-annealing step for the second oligonucleotide primer, and the annealed second primer with the first reverse complementary oligonucleotide from said pre-annealing step for said second oligonucleotide primer, wherein said step of heating the amplification solution for a chosen time at a single chosen temperature, further comprises the second oligonucleotide primer separating from the annealed second oligonucleotide primer with the second reverse complementary oligonucleotide from said pre-annealing step for the second primer such that said RT-LAMP amplification reaction takes place, and said step of cooling the amplification solution following said RT-LAMP amplification reaction further comprises unreacted second reverse complementary oligonucleotides being again annealed to unreacted second oligonucleotide primers.

7. The method of claim 6, wherein the second oligonucleotide primer comprises the FIP for hybridizing with the human RNase P POP7 gene nucleic acid control molecule.

8. The method of claim 7, wherein the second oligonucleotide primer has a sequence of SEQ ID NO: 7, and the second reverse complimentary oligonucleotide has a sequence of SEQ ID NO: 14.

9. The method of claim 6, wherein the second fluorophore comprises FAM, and the second fluorescence quencher is chosen from BHQ1, lowaBlack-FQ, and TAMRA.

10. The method of claim 1 further comprising the step of storing the aqueous solution from said step of preparing an aqueous solution for a chosen period of time before said step of adding the heat-treated, stabilized saliva to the aqueous solution.

11. The method of claim 1, wherein the chemical stabilizer for saliva comprises a proteinase enzyme stabilizer.

12. The method of claim 1, wherein the aqueous solution further comprises deoxyuridine triphosphate.

13. The method of claim 1, wherein the aqueous solution further comprises Antarctic thermolabile uracil DNA glycosylase.

14. The method of claim 1, wherein said step of heating the amplification solution is performed for about 45 min. and the single chosen temperature is about 65° C.

15. The method of claim 1, wherein said step of cooling the amplification solution following said RT-LAMP amplification reaction is performed for about 5 min. at a temperature of about 21° C.

16. The method of claim 1, wherein said step of observing the fluorescence radiation is performed by visual inspection, a reddish color indicating the presence of SARS-CoV-2 virus, and a greenish color indicating the absence of the SARS-CoV-2 virus.

17. A method using RT-LAMP amplification for detecting nucleic acid from SARS-CoV-2, comprising the steps of:
collecting a sample of raw saliva;
providing a chemical stabilizer, wherein the sample is mixed with the chemical stabilizer, forming stabilized saliva;
heating the stabilized saliva for a chosen time at a chosen temperature to inactivate the chemical stabilizer, forming heat-treated, stabilized saliva;
pre-annealing a first FIP oligonucleotide primer for hybridizing SARS-CoV-2 N gene nucleic acid having SEQ ID NO: 1, the first FIP oligonucleotide primer being conjugated to a first fluorophore at its 5′ end, with a first fluorescence quencher for the first fluorophore conjugated to the 3′ end of a first reverse complementary oligonucleotide sequence to the first FIP oligonucleotide primer having SEQ ID NO: 13, forming an annealed first FIP primer with a first reverse complementary oligonucleotide; and pre-annealing a second FIP oligonucleotide primer for hybridizing human RNase P POP7 gene nucleic acid having SEQ ID NO: 7, the second oligonucleotide primer being conjugated to a second fluorophore at its 5′ end, with a second fluorescence quencher for the second fluorophore conjugated to the 3′ end of a second reverse complimentary oligonucleotide sequence to the second FIP oligonucleotide primer having SEQ ID NO: 14, forming an annealed second FIP primer with a second reverse complementary oligonucleotide;
preparing an aqueous solution comprising: DNA polymerase, reverse transcriptase, deoxyadenosine triphosphate, deoxycytidine triphosphate, deoxyguanosine triphosphate, deoxythymidine triphosphate, BIP, F3, B3, Loop F, and Loop B first oligonucleotide primers for hybridizing with the SARS-CoV-2 N gene nucleic acid sequence, BIP, F3, B3, Loop F, and Loop B second oligonucleotide primers for hybridizing with the human RNase P POP7 gene nucleic acid control, the annealed first FIP primer with the first reverse complementary oligonucleotide from said pre-annealing step for the first FIP primer, the annealed second FIP primer with the second reverse complementary oligonucleotide from said pre-annealing step for said second FIP primer, magnesium sulfate, betaine, Tris(hydroxymethyl)aminomethane hydrochloride, ammonium sulfate, potassium chloride, and Tween 20;
adding the heat-treated, stabilized saliva to the aqueous solution, forming an amplification solution;
heating the amplification solution for a chosen time at a single chosen temperature, whereby the first FIP primers are separated from the annealed first FIP primers with the first reverse complementary oligonucleotide from said pre-annealing step for the first FIP primers, and whereby the second FIP primers are separated from the annealed second FIP primers with the second reverse complementary oligonucleotides from said pre-annealing step for the second FIP primers, such that said RT-LAMP amplification reaction takes place;
cooling the amplification solution following said RT-LAMP amplification reaction for a chosen period of time at a chosen temperature, forming a cooled amplification solution, whereby unreacted first reverse complementary oligonucleotides are again annealed to unreacted first FIP primers, and unreacted second reverse complementary oligonucleotides are again annealed to unreacted second FIP primers;
providing an ultraviolet light, wherein the cooled amplification solution is illuminated using the ultraviolet light, and wherein the ultraviolet light has a chosen wavelength such that incorporated fluorophore-conjugated oligonucleotides from said RT-LAMP amplification reaction emit fluorescence radiation; and
observing the fluorescence radiation.

18. The method of claim 17, wherein the first fluorophore comprises Texas Red, and the first fluorescence quencher is chosen from BHQ2, lowaBlack-RQ, and BlackBerry Quencher 650.

19. The method of claim 17, wherein the second fluorophore comprises FAM, and the second fluorescence quencher is chosen from BHQ1, lowaBlack-FQ, and TAMRA.

20. The method of claim 17 further comprising the step of storing the aqueous solution from said step of preparing an aqueous solution for a chosen period of time before said step of adding the heat-treated, stabilized saliva to the aqueous solution.

21. The method of claim 17, wherein the chemical stabilizer comprises a proteinase enzyme stabilizer.

22. The method of claim 17, wherein the aqueous solution further comprises deoxyuridine triphosphate.

23. The method of claim 17, wherein the aqueous solution further comprises Antarctic thermolabile uracil DNA glycosylase.

24. The method of claim 17, wherein said step of incubating the amplification solution is performed for about 45 min. and the single chosen temperature is about 65° C.

25. The method of claim 17, wherein said step of cooling the amplification solution following said RT-LAMP amplification reaction is performed for about 5 min. at a temperature of about 21° C.

26. The method of claim 17, wherein said step of observing the fluorescence radiation is performed by visual inspection, a reddish color indicating the presence of SARS-CoV-2 virus, and a greenish color indicating the absence of the SARS-CoV-2 virus.

27. An oligonucleotide for conjugation with a quencher, wherein said oligonucleotide has SEQ ID NO: 13.

28. The oligonucleotide of claim 27, wherein said quencher is chosen from BHQ2, lowaBlack-RQ, and BlackBerry Quencher 650.

29. An oligonucleotide for conjugation with a quencher, wherein said oligonucleotide has SEQ ID NO: 14.

30. The oligonucleotide of claim 27, wherein said quencher is chosen from BHQ1, lowaBlack-FQ, and TAMRA.

31. A kit for detecting nucleic acid from SARS-CoV-2 by RT-LAMP amplification, comprising:
at least one screw-capped saliva sample tube having a first chosen volume and containing a dried stabilization component;
at least one fixed-volume micropipette, or at least one fixed-volume capillary tube having a plunger;
at least one optically clear reaction tube having a second chosen volume, each tube having an attached snap cap, and containing a selected volume of the aqueous solution comprising: DNA polymerase, reverse transcriptase, deoxyadenosine triphosphate, deoxycytidine triphosphate, deoxyguanosine triphosphate, deoxythymidine triphosphate, BIP, F3, B3, Loop F, and Loop B first oligonucleotide primers for hybridizing with the SARS-CoV-2 N gene nucleic acid sequence, BIP, F3, B3, Loop F, and Loop B second oligonucleotide primers for hybridizing with the human RNase P POP7 gene nucleic acid control, an annealed first FIP primer conjugated to a first fluorophore and having SEQ ID NO: 1, with a first reverse complementary oligonucleotide conjugated to a first quencher for the first fluorophore and having SEQ ID NO: 13, an annealed second FIP primer conjugated with a second fluorophore and having SEQ ID NO: 7, with a second reverse complementary oligonucleotide conjugated to a second quencher for the second fluorophore and having SEQ ID NO: 14, magnesium sulfate, Tris(hydroxylmethyl)aminomethane hydrochloride, ammonium sulfate, potassium chloride, and Tween 20;
a dry heating block or a water bath capable of being heated, for heating the at least one clear reaction tube to about 65° C., and the at least one saliva sample tube to ≥95° C.;
an ultraviolet lamp; and
an ultraviolet filter for viewing fluorescence.

32. The kit of claim 31, further comprising: 3 control reactions samples for visual comparison of fluorescence results, comprising: (a) a positive control reaction containing both human and SARS-CoV-2 RNA; (b) a negative control reaction containing only human RNA; and (c) a no-reaction control containing no RNA.

33. The kit of claim 31, wherein the aqueous solution further comprises deoxyuridine triphosphate.

34. The kit of claim 31, wherein the aqueous solution further comprises Antarctic thermolabile uracil DNA glycosylase.

35. The kit of claim 31, wherein the aqueous solution further comprises Bentaine.
